(19) United States
(12) Patent Application Publication
(10) Pub. No.: US 2025/0298018 A1
(43) Pub. Date: Sep. 25, 2025

(54) TEST FOR THE DETECTION OF ANTIBODIES AGAINST LEISHMANIA

(71) Applicant: Philipps-Universität Marburg, Marburg (DE)

(72) Inventors: Ulrich Steinhoff, Marburg (DE); Rouzbeh Mahdavi, Marburg (DE)

(21) Appl. No.: 18/730,524

(22) PCT Filed: Jan. 18, 2023
(86) PCT No.: PCT/EP2023/000003

§ 371 Date: Jul. 19, 2024

(30) Foreign Application Priority Data
Jan. 20, 2022 (EP) 22152398.8

(51) Int. Cl. G01N 33/569; C07K 14/44

(52) U.S. Cl. CPC G01N 33/56905; C07K 14/44; G01N 2333/44; G01N 2469/20; G01N 2470/04

(57) ABSTRACT

The invention relates to a method for detecting antibodies against Leishmania in a biological sample. The invention further relates to a test for carrying out the method, the production of the test antigen, and the test antigen with SEQ ID NO. 8.

The invention relates to a method for detecting antibodies against Leishmania in humans and dogs (patients). A novel test antigen is used for the new serological method. The disease leishmaniasis is diagnosed by detecting antibodies against Leishmania. The novel test antigen binds specifically to antibodies that a patient has formed after an infection with Leishmania. This test antigen-antibody binding is subsequently visualized using a suitable method known to those skilled in the art, for example using a labeled secondary antibody. If a patient has been infected with Leishmania and is suffering from leishmaniasis, this patient has also formed antibodies against Leishmania, which can be detected using a serological method. The invention relates to a novel test antigen with which antibodies against Leishmania can be detected across species (i.e., in humans and dogs as patients) and across strains (i.e., in infections with different Leishmania strains or Leishmania species). In particular, the invention relates to a method that may be carried out using a point-of-care test (POCT, near-patient laboratory diagnostics, rapid test) for direct application to humans and dogs in countries affected by leishmaniasis.

The invention further relates to the production of this novel test antigen and a new serological test (point-of-care test, POCT, laboratory diagnostics close to the patient, rapid test) for the serological detection of antibodies against Leishmania, using the novel test antigen.

Leishmaniasis is caused by parasitic protozoa from the genus Leishmania. The clinical symptoms may be very diverse, depending on which organ is primarily affected. Without treatment, leishmaniasis can be fatal, so that early diagnosis is crucial in order to initiate targeted therapy. A distinction is made between a visceral form of leishmaniasis (visceral leishmaniasis (VL), kala azar, dum dum fever, black fever), a cutaneous form (cutaneous leishmaniasis (CL), skin leishmaniasis, Baghdad bubo, oriental bubo, Aleppo bubo), and a mucocutaneous form (mucocutaneous leishmaniasis (MCL), mucosal leishmaniasis, uta, espundia). The World Health Organization (WHO) has declared visceral leishmaniasis (VL) to be one of the most neglected diseases, and is therefore promoting diagnostics and therapy in a special way. In addition to humans, Leishmania can also infect animals, especially dogs, as well as cats, rodents, cattle, horses, and guinea pigs.

A large number of different Leishmania strains are known, for example L. chagasi, L. donovani, L. infantum, L. major, L. braziliensis, L. archibaldi, and L. tropica. Individual Leishmania strains prefer different hosts, and cause different clinical symptoms after infection.

Kinesins are a superfamily of motor proteins found in all eukaryotes. In Leishmania, they play an important role in regulation of the length of the flagellum, cell division, intracellular transport of various proteins, and formation of cytoskeletal filaments. The kinesin is highly conserved with regard to the nucleotide, and thus also the amino acid sequence, and shows an identity of 80 to 90% between different Leishmania strains. The K39 kinesin, also known as LcKin, is a Leishmania kinesin, and was originally identified by screening an expression library of Leishmania infantum (synonymous with Leishmania chagasi) with sera from patients with visceral leishmaniasis.

Such patients show a strong antibody response to the kinesin, which consists of a repetitive repeat of 39 amino acids. Therefore, kinesins are suitable as a diagnostic antigen (test antigen for serological tests).

Leishmania is transmitted mainly by sand flies (Phlebotominae) from the moth fly family (Psychodidae). Due to increasing global warming and globalization, the sand flies are spreading primarily northwards, increasing the risk of infection with Leishmania even in countries that so far have experienced little or no infection. Intensive contact with infected pets, especially dogs, also increases the risk of leishmaniasis infection in humans. In this case, the leishmaniasis is a zoonosis.

Since leishmaniasis occurs mainly in tropical and subtropical countries, and in dogs also in Mediterranean countries, the distribution, storage, and use of test systems for detecting antibodies against Leishmania is a major challenge. There is currently no sufficiently reliable test procedure, test system, or test kit that mobile medical test personnel can use to carry out reliable Leishmania diagnostics in the various endemic areas, which are characterized by different occurrences of the pathogen. As a result, many patients remain undiagnosed and therefore untreated, especially since the clinical symptoms of leishmaniasis are often very similar to the symptoms of other tropical diseases such as malaria or schistosomiasis (bilharzia).

### Prior Art

There are two basic options for diagnosing leishmaniasis: either the pathogen (i.e. the Leishmania) or portions of the pathogen (for example, its DNA or certain surface structures) is/are detected directly in a patient, or antibodies against Leishmania, which the patient has formed as the result of infection with Leishmania, are detected. In the first case, the disease is indicated by detection of the pathogen or components of the pathogen. In the second case, the disease is indicated by the antibody detection.

Direct detection of the pathogen or components of the pathogen encompasses microscopic detection of the pathogen, PCR, and antigen detection. The antigen detection, the same as the detection of antibodies, is based on the specific binding of antibodies (which the patient has formed against Leishmania) to the antigen (originates from Leishmania or was derived from Leishmania). This binding is subsequently visualized using suitable methods. The reliability of the antigen or antibody detection depends essentially on the quality of the test antigen and the antibodies used. Since the antibodies to be detected are usually detected in a patient's blood serum, this test method is referred to as a serological test (immunoassay, immunosorbent assay (ISA)) or serological diagnostics. Examples of such serological tests are the ELISA, the radioimmunoassay, the enzymatic immune adsorption test (EIA), or the immunoblot.

The following direct, serological methods are currently available for diagnosing leishmaniasis:
Direct detection of the pathogen in a biological sample (tissue sample, biopsy, swab preparation, imprint preparation, aspirate, blood smear, buffy coat preparation): Leishmania can be detected in its amastigote form (as Leishman-Donovan bodies) in white blood cells, in bone marrow cells, in spleen cells, in lymph node cells, in liver cells, or in skin cells by microscopic examination after staining with a suitable dye (for example, Giemsa stain, Wright or Leishman stain). For this purpose, trained medical staff in a hospital or a specialized medical practice must take the relevant tissue samples from the patient. This medical procedure may be uncomfortable, painful, or even dangerous for the patient. Tissue sampling may require an ultrasound machine to find the right biopsy site. The tissue sample must then be prepared and stained. This requires appropriate laboratory equipment and trained specialists. Finally, the tissue sample must be examined under a microscope. Obtaining a reliable result requires considerable experience as well as a suitable microscope. If the Leishmania to be detected are in poorly stained or too strongly stained areas of the tissue sample or are covered by other structures, then detection is hardly possible. The direct microscopic detection of pathogens therefore has the following disadvantages: Potential damage to the patient when obtaining the tissue sample, high technical outlay and expenditure of labor, the need for a fully equipped laboratory, and a high degree of dependence on the experience of the examiner. Consequently, subjective errors with resulting false negative results are common. In addition, the direct detection of the pathogen is comparatively expensive and time-consuming and provides only limited information. Furthermore, a negative result of the examination is of little significance, since when the tissue sample is taken, a spot by chance may be found in which there are no Leishmania, and the Leishmania present in a tissue sample may be overlooked.
Pathogen detection using PCR (PCR-based assay): The polymerase chain reaction (PCR) is an established method for detecting Leishmania DNA in a patient's biological sample. Thus, it is not the pathogen itself that is detected, but, rather, its DNA after it has been duplicated in the laboratory. Leishmaniasis diagnostics using PCR have the following disadvantages: They take a relatively long time, require specific reagents that must be constantly cooled, are relatively expensive, and require complex laboratory equipment and personnel with extensive PCR experience. This means that a PCR cannot be carried out in the field. A further, PCR-specific disadvantage is that the smallest errors in the implementation may very easily lead to false positive results, for example in the case of laboratory contamination. However, false negative results are also common with a PCR if, for example, the quantity of DNA present is too small or if inhibitors against DNA are present in the biological sample.
Detection of Leishmania antigens in a biological sample (tissue) using serological test methods: If a patient is infected with Leishmania, components of the Leishmania, such as surface structures, may be bound with suitable test antibodies. This binding is subsequently visualized using suitable methods, for example using a secondary antibody. A suitable biological sample (a tissue sample, for example) may be taken, analogously to the direct microscopic detection of the pathogen. This method has the following disadvantages: Potential damage to the patient when obtaining the tissue sample, and the requirement for one or more Leishmania strain-specific test antibodies. In addition, a negative result of the examination is of little significance, since when the tissue sample is taken, a spot by chance may be found in which no Leishmania antigens are present. The sensitivity of this method is low in patients with a suspected clinical diagnosis. In the prior art there is a commercially available latex agglutination test (KAtex test from Kalon Biological Ltd.) with which 5-20 kDa glycoproteins in the urine of patients are detected. The sensitivity of this test is low, especially when detecting Leishmania in India and East Africa. The specificity of this test is low in immunocompetent individuals, and a large number of false positive results occur. 25
Enzyme-linked immunosorbent assay (ELISA): The ELISA is an enzymatic immunosorbent method (EIA) via which proteins or polypeptides (antibodies or antigens, for example) can be detected. It is likewise based on the specific binding between an antigen and the corresponding antibody. Antibody detection using ELISA has the following disadvantages: It takes a relatively long time, requires specific reagents, is therefore relatively expensive, and requires complex laboratory equipment as well as trained personnel. Another disadvantage of the ELISA is the determination of the correct cut-off point via which a negative signal is distinguished from a positive signal. This is particularly relevant for patients with a low Leishmania antibody titer (for example, due to an immunodeficiency as the result of infection with the human immunodeficiency virus (HIV) or other co-infections.
Radioimmunoassay (RIA): In an RIA, the specific binding between an antigen and the corresponding antibody is detected by coupling the antigen with a radioactive substance. By measuring the radioactivity, conclusions may be drawn concerning the presence or quantity of the antibody in the sample. The detection of pathogens using RIA has the following disadvantages: It takes a relatively long time, requires specific reagents, is therefore relatively expensive, and requires very complex laboratory equipment as well as specifically trained personnel. In particular, radioactive substances are necessary, the production, storage, and disposal of which are very problematic. A specific disadvantage of all nuclear medicine in vitro test methods is the occurrence of signals that are too low despite a high concentration of the antigen (high-dose hook effect).
Immunoblot (IB): The immunoblot is an immunoassay method with a spatially separate fixation of the specified antibodies. Examples of an immunoblot are the western blot, the dot blot, or the slot blot. The test principle of an immunoblot is also used for rapid tests (POCTs), for example as a lateral flow assay (LFA). An immunoblot in its original form has the following disadvantages: It is very complicated and time-consuming, and requires very complex laboratory equipment as well as specifically trained personnel. The costs for an immunoblot are therefore also very high.
Direct agglutination test (DAT): The DAT is an immunochromatographic method which can be carried out only in well-equipped laboratories, and takes about 10 hours. The DAT has the following disadvantages: It takes a relatively long time, requires specific reagents, is therefore relatively expensive, and requires very complex laboratory equipment as well as specifically trained personnel. The test antigen required for the DAT must be stored at 4-8° C., which cannot be guaranteed in tropical and subtropical countries with a deficient infrastructure.
Antigen-coated dipstick test: The dipstick test is likewise an immunochromatographic method that is available as a rapid test. Blood or blood serum may be used as sample material. The dipstick test may be performed on a test strip coated with a Leishmania-specific test antigen. If the sample material contains antibodies against Leishmania, they bind to the test antigen. There are commercially available immunochromatographic strip tests (ICTs) in the prior art that have the known test antigen rK39 (Kalazar Detect™ from InBios International, Inc. and Onsite Leishmania Ab Rapid Test from CTK Biotech, Inc., both as dipstick tests). rK39 is a recombinant kinesin antigen from Leishmania infantum that contains 6.4 repetitive regions, each with 39 amino acids (repeats). The disadvantage of the rK39 test antigen is that it has a greatly different sensitivity and specificity, depending on the Leishmania strain and the region of origin. The sensitivity and specificity of rK39 in patients in East Africa is therefore very low. This means that the reliable use of rK39-based test systems is limited to only certain countries.

In all serological methods, the quality of the test antigen is critical for the detection of antibodies: The specificity and sensitivity of a serological test depend on the specific binding of an antibody to the corresponding antigen; i.e., the poorer the accuracy of fit between an antigen and antibodies, the poorer the sensitivity and specificity of the test performed. A fundamental problem with serological tests is the occurrence of cross-reactions. False-positive results occur when the test antigen or test antibody used reacts non-specifically.

Commercially available tests for the serological diagnosis of leishmaniasis are based either on an antigen from Leishmania infantum, the kinesin protein rK39, or on an antigen from Leishmania donovani, the kinesin protein rKE16. The test antigen rK39 is currently the most widely used recombinant antigen for the serological diagnosis of leishmaniasis in East African countries. In India, the test antigen rKE16 is primarily used because it is superior to rK39 here. Since the incidence of visceral leishmaniasis is highest in the world in East Africa, but at the same time serodiagnostics have been very unreliable here, there is an urgent need for a universally usable test antigen with the highest diagnostic performance (sensitivity and specificity) that is independent of the endemic area.

### Object of the Invention

The object of the invention, therefore, is to provide an improved method for the detection of Leishmania-specific antibodies across species (humans/dogs) and across Leishmania strains. The test antigen required for this purpose should show a very high sensitivity and specificity for all strains that cause visceral leishmaniasis; i.e., the method should be usable independently of the endemic area. The test antigen required for this purpose should also detect antibodies against Leishmania in human biological samples (in serum, for example) as well as in biological samples from dogs (canine serum). The improved method should be suitable for worldwide use to detect antibodies against Leishmania in humans and dogs. A suitable test for this method is described. A method for producing this novel test antigen is also described. The test antigen should not show any cross-reactivity with malaria, tuberculosis, or other co-infections. Likewise, the test antigen should be stably storable over an extended time period (several years) without losing its biological functions.

### Achievement of the Object

The present invention achieves the object by providing the novel, artificially produced test antigen rKLi8.3, which has an immunologically active amino acid sequence as shown in SEQ ID NO. 8 and is used in the method according to the invention. The SEQ ID NO. 7 is the nucleic acid sequence of the test antigen according to the invention, and the SEQ ID NO. 8 is the corresponding amino acid sequence. The abbreviations for the nucleic acids and the amino acids are known to those skilled in the art. This artificially produced test antigen reacts with antibodies against various Leishmania strains, both in humans and in dogs. The binding of the novel, artificially produced test antigen with antibodies from Leishmania-infected humans or dogs shows a very high specificity and sensitivity.

This is demonstrated in scientific studies by the inventors. The antibody-test antigen binding of the novel, artificially produced test antigen with the human or dog antibodies to be detected is visualized according to one of the known methods using a known standard procedure, for example with a color reaction (chromogenic, colorimetric methods, chemiluminescence, fluorescence, or by means of gold colloid). In the positive case, i.e., if antibodies are detected, it is concluded that the patient to be tested (human/dog) is infected with Leishmania. The present invention also encompasses a test (test system, test kit, rapid test) using the novel, artificially produced test antigen.

However, the currently available serological tests show different diagnostic sensitivity and specificity in different endemic areas, for which reason infections with Leishmania often cannot be reliably diagnosed using a test antigen. The reason for this may be the strain-specific sequence (kinesin sequences vary in different Leishmania strains), the structure of the kinesin test antigen (number of kinesin repeats), or the combination of both. The inventors have systematically analyzed these aspects. The result of their studies is that, in addition to sequence variability, the kinesin antigen structure (number of repeats) has a dominant influence on the diagnostic sensitivity and specificity.

For this purpose, kinesin proteins with an increasing number of repeats from different Leishmania strains were recombinantly produced and tested for the binding strength of antibodies from patients with visceral leishmaniasis (VL). It was shown that the antibody binding to the kinesin test antigen is stronger as the number of repeats increases. The inventors were thus able to demonstrate that the number of repeats increases the affinity between the test antigen and the antibody. Recombinant kinesin proteins from various Leishmania strains with different sequences but the same number of repeats were subsequently analyzed. It was shown that, in addition to the number of repeats, the amino acid sequence of the kinesins also has an influence on antibody binding. It was shown here that highly conserved kinesin proteins, which have hydrophilic properties, react best with the patient's antibodies and are therefore best suited as test antigens.

When test antigens are cloned, repeated DNA sequences (tandem repeats), such as kinesin here, are usually very difficult to clone and express. Working with repetitive DNA presents many challenges. The amplification of multiple tandem repeats by means of PCR is difficult, since shorter fragments are preferentially amplified, and long sequence regions are transcribed and translated very inefficiently.

In addition, increased recombination or chimera formation may occur as the result of the PCR amplification. Molecular mechanisms resulting in the generation of such recombination are mostly unknown or unclear. In bacteria, repetitive DNA sequences are often recombined or eliminated. In addition, toxic effects often occur in bacteria that have been transformed with repetitive sequences, for which reason the desired protein is expressed only incompletely or not at all.

Since a novel test antigen had to be developed for the method according to the invention, the inventors first dealt with the development of a novel, improved test antigen.

The present invention also encompasses the production of the novel test antigen from selected Leishmania strains by means of PCR, and cloning in competent E. coli bacteria. The following steps are carried out for this purpose:
(i) providing promastigote Leishmania;
(ii) isolating the genomic DNA from the promastigote Leishmania from step (i);
(iii) amplifying the gene segment from the DNA from step (ii) according to SEQ ID NO. 3 with the forward PCR primer according to SEQ ID NO. 1 and the reverse PCR primer according to SEQ ID NO. 2, resulting in an amplification product;
(iv) isolating and purifying the amplification product from step (iii);
(v) cloning the purified amplification product from step (iv) into a vector;
(vi) transfecting competent E. coli with the vector from step (v);
(vii) expressing the test antigen in E. coli;
(viii) lysing the E. coli from step (vii);
(ix) isolating the test antigen from the lysed E. coli from step (viii);
(x) purifying test antigen from step (ix).

The production steps are described in greater detail:
culturing promastigote Leishmania in cell culture medium RPMI-1640 with L-glutamine, NaHCO₃, and 10% (v/v) fetal calf serum (FCS)
isolating genomic DNA from cultured promastigote Leishmania
preparing the forward and reverse PCR primers: The forward primer has the SEQUENCE ID NO. 1 and the reverse primer has the SEQUENCE ID NO. 2. The amplified gene segment has the SEQUENCE ID NO. 3. The kinesin antigen is located on chromosome 14 of Leishmania. This antigen is part of the Leishmania kinesin protein. Within the scope of the inventive activity, highly conserved kinesin sequences from seven East African Leishmania isolates were determined by culturing, cloning, and sequencing the Leishmania, and comparing them to previously published kinesin sequences to identify a kinsesin test antigen that could be used universally and ubiquitously (regardless of the Leishmania strain and endemic area). These analyses led to the rational selection (identification) of an optimal (strain-independent, i.e., Leishmania isolate-independent) antigen arising from the amplification of 8.3 repeats in L. infantum from East Africa.

The further inventive activity after the identification of the strain-independent test antigen involves the following methodology:
carrying out the PCR reaction
isolating the amplification product of rKLi8.3 with 8.3 repeats and 1117 base pairs from the gel
cloning the purified amplification product into a vector, for example the pCR®2.1-TOPO vector (Invitrogen)
transforming competent E. coli with the vector from the previous step
expressing the recombinant test antigen rKLi8.3 in E. coli
destroying (breaking down, lysing) the E. coli, for example by means of a microfluidizer, to release the recombinant test antigen rKLi8.3
purifying the recombinant test antigen rKLi8.3 expressed in E. coli from the bacterial lysate from the previous step, for example by means of Ni affinity chromatography and subsequent size exclusion chromatography.

In order to produce the test antigen for the method according to the invention, the following difficulties had to be overcome when cloning the highly repetitive sequences:
The DNA of the kinesin genes has a very high GC content (>65%). A high GC content alters the secondary structure and increases the stacking interactions, for which reason the amplification of such fragments by the polymerase chain reaction (PCR) often results in undesired products.

Therefore, the inventors have systematically investigated various aspects of how such undesired recombination occurs and how it can be eliminated. These issues were resolved as follows:
optimization of the PCR program
use of different annealing times
use of different annealing temperatures
use of different denaturation temperatures
use of primers with different annealing sites
use of different DNA concentrations
use of competent recA knockout E. coli: RecA is a 38 kilodalton protein in E. coli that plays a role in DNA repair and maintenance. RecA (E. coli) possesses a repair system that recombines homologous sequences. The amplification of repetitive eukaryotic DNA sequences in bacteria is problematic due to rearrangement or deletion by DNA repair systems (recA) in E. coli. These rearrangements result in the following disadvantageous effects:
deletion or duplication of genes flanked by direct sequence repeats
a change in the number of repeating elements
recombination events

In order to overcome these negative effects, special strains of bacteria suitable for cloning the unstable, repetitive DNA were used. These strains carry mutations in their recombinase genes (for example, recA1, recB, recA13). recA13 and recA1 can reduce the recombination of cloned DNA and are therefore suitable for cloning larger numbers of tandem repeats. It was possible to determine that highly repetitive DNA requires a high denaturation temperature so that the double strand with tandem sequences is efficiently opened and the polymerase can dock well.

It was also determined that the binding of the primer to the DNA (annealing) does not necessarily begin at the 3′ end, but, rather, may take place over the entire length of the strand due to the repetitive structure (repeats). Therefore, by extending the nonrepetitive pre-repeat, it was possible to achieve position-defined annealing of the primers.

In order to carry out the method according to the invention, a biological sample of the patient to be tested (humans or dogs) must be made available beforehand. The biological sample is usually serum (blood serum), but it may also be whole blood, plasma (blood plasma), lymph node aspirate, saliva, tissue fluid, cerebrospinal fluid, urine, or some other body fluid which in the case of an infection with Leishmania contains antibodies against Leishmania. The method according to the invention for detecting antibodies against Leishmania comprises the following steps:
(i) providing a biological sample of a human or a dog;
(ii) providing the test antigen, bound to a substrate, containing SEQ ID NO. 8, which has 8.3 repetitive sequences and which can be recognized and bound by antibodies against various Leishmania strains;
(iii) incubating the biological sample from step (i) with the test antigen from step (ii), so that antigen-antibody complexes are formed if the biological sample from step (i) contains antibodies against at least one Leishmania strain;
(iv) removing antibodies and other material, not bound to the test antigen from step (ii), from the biological sample from step (i) in a first washing step;
(v) adding a human-specific secondary antibody if the biological sample from step (i) comes from a human, or adding a canine-specific secondary antibody if the biological sample from step (i) comes from a dog, wherein the respective secondary antibody is detectable using an agent for visualizing binding of the secondary antibody, so that the respective secondary antibody binds to the antigen-antibody complexes from step (iii);
(vi) removing unbound secondary antibodies from step (v) in a second washing step;
(vii) visualizing the binding of the secondary antibody to the antigen-antibody complexes from step (iii) by adding a suitable agent.

The binding of the antibodies to be detected to the test antigen and the visualization of this antibody-test antigen binding may be carried out using known methods, for example by means of ELISA, immunochromatographic methods, or a rapid test (dipstick, line blot). The materials and methods required for this purpose are known to those skilled in the art.

In one preferred exemplary embodiment, antibody-test antigen binding is detected using an anti-human secondary antibody. An example of such is the peroxidase-conjugated donkey anti-human IgG (H+L) from Jackson Immunoresearch Laboratories, US.

In another preferred exemplary embodiment, antibody-test antigen binding is detected using an anti-canine secondary antibody. An example of such is the peroxidase-conjugated rabbit anti-dog IgG (H+L) from Jackson Immunoresearch Laboratories, US.

In another exemplary embodiment, the antibody-test antigen binding may be detected using any secondary antibody directed against human IgM/IgG.

In another exemplary embodiment, the antibody-test antigen binding may be detected using any secondary antibody directed against canine IgM/IgG.

In one exemplary embodiment, the method according to the invention is implemented as a dipstick test (strip test, rapid test, ICT). In this immunochromatographic method, blood or blood serum may be used as the sample material. The dipstick test is carried out on a test strip that is coated with the test antigen according to the invention. If the biological sample contains antibodies against Leishmania, they bind to the test antigen according to the invention. This antibody-test antigen binding is visualized by an immunochromatographic method known to those skilled in the art.

If the method according to the invention is not carried out as an ELISA, but, rather, as a dipstick test, line blot, or lateral diffusion test, the method according to the invention must be modified in a manner known to those skilled in the art, for example by eliminating the washing steps or entraining a negative control.

### EXEMPLARY EMBODIMENTS

The exemplary embodiments explain the inventive activity of the inventors which has led to the novel test antigen.

Proceeding from the limited sensitivity and specificity of the known test antigens, the inventors searched for an improved test antigen. Using different Leishmania isolates, they examined the influence of the antigen structure (influence of the number of repeats) and the antigen sequence (influence of sequence variations) on antigenicity and cross-strain conservation. For this purpose, the inventors cloned, sequenced, and bioinformatically analyzed more than 53 kinesin fragments of various Leishmania species and strains from Sudan. All amino acid variations of the 53 analyzed kinesin fragments from seven isolates (L. archibaldi two times; L. infantum two times, and L. donovani three times) are summarized in FIG. 6. First, a nonrepetitive sequence, the pre-repeat of kinesin, was compared (46 amino acids). It was shown that the amino acid sequence of the pre-repeats of different Leishmania strains is highly conserved. Only at position 41 does the sequence vary between the amino acids cysteine and serine. In the 46aa non-repeating region, the only deviation from L. infantum (L. chagasi) rK39 and the KE16 from L. donovani was Cys→Ser⁴¹.

The inventors show that East African sequences have multiple sequence variations with respect to rK39. Most variations within the 5′ repeat region are not conserved, with several substitutions accompanied by changes in charge, while the amino acid substitutions in the 3′ half of the repeats were mostly conserved. When the rK39 amino acid sequence was compared to 53 constructs containing kinesin fragments from East African strains, positions 4, 6, 16, and 18 were particularly affected by substitutions associated with changes in charge. For all strains from Sudan, the diversity in the first half of each kinesin repeat was particularly striking, as they contain charged amino acids at the above-mentioned positions. Such changes in charge may reduce the antigenicity of the kinesin protein. The inventors can predict that the diagnostic epitopes lie particularly in the second half of the kinesin repeat, since this segment (amino acids 28-34) is completely conserved in all isolates examined.

The following polymorphisms with respect to rK39 were identified in East African isolates: Gln→Leu⁴, Gln→Arg⁴, Arg→Leu⁶, Ser→Leu⁸, Ala→Gly¹³, Ala→Lys¹⁶, Ser→Ala¹⁶, Eqn→Equ¹⁸, and Met→Thr²⁷.

The inventors have found that there is a large genetic diversity of kinesins between Leishmania strains from East Africa, India, and Brazil. This heterogeneity of kinesin antigens explains why rK39 (Brazil) and rKE16 (India) underperform in Africa.

The gene fragment encoding the immunodominant repeats of L. infantum (strain MHOM/SD/82/GILANI) from Sudan, referred to herein as KLi8.3, was amplified from genomic DNA from promastigote Leishmania. The Leishmania were cultured in medium RPMI-1640 with L-glutamine, NaHCO₃(Sigma-Aldrich), and 10% (v/v) FCS (Sigma-Aldrich), and genomic DNA was prepared according to standard protocols.

The forward (5′-GAGCTCGCAACCGAGTGGGAGG-3′) and reverse (5′-GCTCCGCAGCGCGCTCC-3′) PCR primers (SEQ ID NOS. 1 and 2) were designed according to the published Leishmania infantum JPCM5 putative kinesin K39 (gene bank: XM_001464261.2). The PCR reaction was carried out using Phusion® High-Fidelity DNA polymerase (FINNZYMES OY, Finland). The reaction was carried out in a total of 50 μL containing 5% (v/v) DMSO, 4 mM MgCl₂, 10 μL GC buffer, 10 mM dNTPs mix (Thermo Scientific), and 200 ng genomic DNA. The PCR was carried out as follows: Denaturation at 98° C. for 30 s, followed by 35 cycles of denaturation at 98° C. for 10 s, annealing at 71.1° C. for 30 s, and extension at 72° C. for 60 s. The amplified products showed several bands of a size corresponding to one to multiple 117 bp repeats. The largest amplification product (1117 bp) was purified from the gel and cloned into a pCR®2.1-TOPO vector (Invitrogen). Competent cells from E. coli bacteria HB101 (Promega) were transformed with the recombinant plasmid pCR2.1/KLi8.3. The cloned sequence was confirmed by restriction digestion with EcoRI (NEB) and by sequence analysis. The sequence is identical to SEQ ID NO. 3.

Tandem repeats of the KLi8.3 sequence were located and displayed using the Tandem Repeats Finder program (SEQ ID NO. 4).

To express the recombinant test antigen, the DNA sequence coding for KLi8.3 was subcloned into the his-tag vector pET28a (+) (EMD Biosciences). The DNA construct pCR2.1/KLi8.3 with the forward primer 5-GTACATATGGAGCTCGCAACCGAGTGGGAGGACGCA-3′ and the reverse primer 5′-TACCTCGAGCAGTGTGCTGGAATTCGCCCTTACTCCGC-AGC-3′ (SEQ ID NOS. 5 and 6) was used. Amplification took place using Phusion Hot Start II DNA Polymerase (Thermofisher Scientific, US) according to the manufacturer's recommendations. The amplified DNA fragments were digested with NdeI and XhoI restriction enzymes and cloned in-frame and downstream from 6×His-tag into the appropriate sites of the vector pET28a (+) to generate the plasmid construct pET28a/KLi8.3. SEQ ID NO. 7 shows the entire DNA construct encoding for the his-KLi8.3 fusion protein. The SEQ ID NO. 7 consists of the SEQ ID NO. 3 with 6×histidine residues encoded by the following nucleotides: CATCATCATCATCATCAC.

The rKli8.3 protein was purified over an Ni-A affinity column, using 6×his residues. The recombinant plasmid was verified by DNA sequencing and restriction analysis, and was subsequently transformed into NEB 5-alpha F′lq Competent E. coli bacteria (New England Biolabs).

The expression of rKLi8.3 in transformed BL21 (DE3) competent E. coli bacteria was induced after addition of 1 mM isopropyl-β-D-1-thiogalactopyranoside (IPTG) and incubation for 3 h at 37° C. and 200 rpm. The cells were lysed in a microfluidizer and the soluble fractions were obtained by centrifugation. The recombinant test antigen was purified by Ni2+ affinity chromatography, using a HisTrap HP 5-mL column (GE Healthcare, US). The last impurities and possible aggregates were removed via size exclusion chromatography. The chromatography was performed using an ÄKTA chromatography system (GE Healthcare, US). The purified rKLi8.3 protein, having 393 amino acids and 6 histidine residues (SEQ ID NO. 8) and a molecular weight of 43.2 kDa, was separated by SDS-PAGE.

Bacterial extracts containing the plasmid pET28a/rKLi8.3 were separated on 12.5% SDS-PAGE and stained with coomassie blue before (0 h) and after (3 h) induction with IPTG. The recombinant test antigen was purified by Ni2+ affinity chromatography, using a His Trap HP 5-mL column (GE Healthcare, US).

The reactivity of the recombinant test antigen rKLi8.3 was examined in a western blot test with 10 pooled sera from patients with confirmed L. donovani infection and 10 pooled control sera from healthy individuals from Sudan. For this purpose, the test antigens were transferred to a nitrocellulose transfer membrane (Whatman GmbH, Germany), using Bio-Rad Semi-dry Trans-Blot at 200 mA for 1 h. The membrane was then blocked with 5% BSA (w/v) in 100 mM NaCl, 0.05% Tween 20 (v/v), and 10 mM tris-HCl, pH 7.4 (blocking buffer), and subsequently incubated at 4° C. for 18 hours with either patient sera or control sera (1:1000 in blocking buffer). After washing, the blots were incubated for 1 hour with peroxidase-conjugated donkey anti-human IgG (H+L) (Jackson Immunoresearch Laboratories, US) (1:10000 dilution) at a temperature between 18 and 25° C. (room temperature, RT) As shown in FIG. 1, the positive serum was recognized by the recombinant test antigen rKLi8.3 (tracks 3 and 4), while the negative serum did not react with the recombinant test antigen rKLi8.3 (track 1). These results confirmed that rKLi8.3 is very well suited as a test antigen for the specific detection of Leishmania antibodies

In one embodiment, the test antigen according to the invention is used in an ELISA for the serological detection of antibodies against Leishmania. The ELISA was performed with MaxiSorp™ high protein binding polystyrene ELISA plates (NUNC™, Serving Life Science, Denmark). First, the protein concentration for coating the plates was analyzed to determine the optimal serum dilutions. For this purpose, pooled sera from 10 characterized Sudanese patients with confirmed visceral leishmaniasis and 10 pooled control sera from healthy people from Sudan were used. Various concentrations of the test antigen according to the invention were titrated against serial dilutions of positive or negative sera. 5 to 50 ng of recombinant test antigen rKLi8.3 per well was coated on ELISA plates overnight at 4° C. in 0.1 M NaCO3 buffer, pH 9.6. The plates were washed with PBS containing 0.05% (v/v) Tween-20, and then blocked with 3% (w/v) BSA in the same buffer at a temperature between 18 and 25° C. (room temperature, RT) for 1 hour. After further washing steps, 50 μL of diluted positive or negative serum samples was added to each well and the plates were incubated for 45 minutes at a temperature between 18 and 25° C. (room temperature, RT). After washing, 50 μL/well of peroxidase-conjugated AffiniPure donkey anti-human IgG (H+L) (Jackson Immunoresearch Laboratories, US) (1:10000) was added to each well, and the plates were incubated for 1 h at a temperature between 18 and 25° C. (room temperature, RT). The reaction was visualized with hydrogen peroxide and tetramethylbenzidine (R&D Systems, US). The reaction was stopped with 2 M sulfuric acid after 10 minutes incubation in the dark. The optical density (OD) was measured at 450/570 nm using an ELISA microreader (FLUOstar Omega, BMG LABTECH). Each sample was tested at least twice, and the mean was calculated. Samples with invalid or conflicting results were repeated.

All tested concentrations (5-50 ng/well) of rKLI8.3 were recognized by the pooled sera from leishmaniasis patients (see FIG. 2). Sera from healthy individuals did not react with the test antigen according to the present invention. The ODs of positive sera were at least 4 times higher than those of negative sera. The coating of ELISA plates with a concentration of 5 ng test antigen was sufficient for the positive detection of sera from Leishmania-infected patients. Therefore, a protein concentration of 5 ng/well and a serum dilution of 1:800 were selected as optimal conditions for detection and used in the subsequent experiments.

The diagnostic performance of all recombinant test antigens known from the prior art was determined using diagnostic efficiency values. The following definitions were used in calculating the corresponding diagnostic parameters: true-positive (tp)=sera from patients with confirmed visceral Leishmania infection; false-negative (fn)=sera from patients with confirmed visceral Leishmania infection with negative values; false-positive (fp)=sera from healthy subjects without visceral Leishmania infection with positive values; true-negative (tn)=sera from healthy subjects without visceral Leishmania infection with negative values; positive predictive value (PPV)=probability that the disease is present if the test is positive, TP/(TP+FP)×100%; negative predictive value (NPV)=probability that the disease is not present if the test is negative, TN/(TN+FN)×100%; sensitivity=tp×100/(tp+fn); specificity=tn×100/(tn+fp). Diagnostic efficacy (DEV) values were calculated from (tn+tp)×100/(tp+fp+tn+fn).

A total of 288 positive or negative human sera were used for the studies. 172 sera were from patients with visceral leishmaniasis (VL) confirmed by lymph node aspiration. 85 sera were from healthy individuals residing in an endemic area for VL. As further controls, sera from Sudanese patients suffering from other common diseases in the endemic areas were included in the study. 5 sera were from patients with confirmed malaria, and 26 sera were from patients with diagnosed tuberculosis. All sera were stored at −20° C. 10 control sera from uninfected German persons were also collected and used.

The ELISA was carried out as described above, using the test antigen rKLi8.3 according to the invention. The patient sera and control sera were diluted 1:800 and assayed with 5 ng rKLi8.3 per well.

The data were analyzed using GraphPad Prism software (GraphPad Prism Inc., San Diego, CA). The healthy controls from Sudan were used to determine the ELISA cut-off value. These were defined as the mean absorbance value of sera from healthy controls plus 3×standard deviations (SD) for each recombinant test antigen.

The recombinant Leishmania chagasi test antigen rk39 known from the prior art, having GenBank accession number AAA29254.1, was purchased from Rekom Biotech, SL, Granada, Spain. It was also expressed as a fusion protein with a 6×his tag at the C-terminus in E. coli. After delivery, the concentration of the test antigen was checked according to the same method used to measure the recombinant test antigens rKLO8 and rKLi8.3 (Bradford). Aliquots were stored at −80° C. Table 1 lists all tested sera together with their origins and properties.

TABLE 1
Sera from patients with visceral leishmaniasis (VL) and controls from Sudan and Germany.

| Origin | Number | | | | |
|---|---|---|---|---|---|
| (country) | of sera | Clinical case | | Properties | Classification |
| Sudan | 288 | VL | (n = 172) | The diagnosis is based on the detection of parasites in | Confirmed VL |
| | | | | stained lymph node smears | patients |
| | | EC | (n = 85) | From the same VL endemic area (Sudan) | Negative controls |
| | | MA | (n = 5) | The diagnosis was performed by detecting malaria parasites | |
| | | | | in blood smears | |
| | | TB | (n = 26) | The diagnosis was performed by detecting AFB of the TB in | |
| | | | | a sputum smear | |
| Germany | 10 | NEC | (n = 10) | Non-endemic healthy volunteers from Germany | |

Abbreviations:
VL, visceral leishmaniasis;
NEC, non-endemic controls;
EC, endemic controls;
MA, malaria;
TB, tuberculosis;
AFB, acid-fast bacilli

The diagnostic performance of the rK39, KLO8, and rKLI8.3 test antigens is presented in

### Table 2.

TABLE 2
Diagnostic performance of the test antigens rK39, KLO8, and rKLi8.3 in the ELISA for visceral leishmaniasis
(VL) in Sudan: AUC = area under curve; TP = true positives; FN = false negatives; TN =
true negative; FP = false positive; PPV = positive predictive value; NPV = negative predictive
value; DEV = diagnostic efficiency value. The specificity was calculated using 126 visceral leishmaniasis-
negative sera, of which 85 were endemic control sera, 10 were non-endemic control sera, 5 were malaria sera,
and 26 were tuberculosis sera. The sensitivity was calculated using 172 visceral leishmaniasis sera. The values
for sensitivity, specificity, PPV, NPV, and DEV were calculated with a 95% confidence interval. The sera were
used at a dilution of 1:800, and the test antigens were used at a concentration of 5 ng. The cut-off value
was determined as the mean absorbance value of the endemic control sera + 3 standard deviations.

| Test antigen | Cut-off | | | | | | | | | | |
|---|---|---|---|---|---|---|---|---|---|---|---|
| (ELISA) | value | AUC | TP | FN | TN | FP | Sensitivity, % | Specificity, % | PPV, % | NPV, % | DEV, % |
| rK39 | 0.146 | 0.9678 | 157 | 15 | 118 | 8 | 91.28 | 93.65 | 95.15 | 88.72 | 92.28 |
| KLO8 | 0.124 | 0.9811 | 159 | 13 | 123 | 3 | 92.44 | 97.62 | 98.15 | 90.44 | 94.63 |
| rKLi8.3 | 0.106 | 0.9927 | 167 | 5 | 125 | 1 | 97.10 | 99.20 | 99.40 | 96.15 | 97.98 |

The quantitative analysis of the test antigen rKLi8.3 with sera from patients with visceral leishmaniasis (VL) showed much higher antibody levels than in the controls. The sera tested with rKLi8.3 test antigen showed higher OD values compared to those tested with the test antigens rK39 and KLO8. The reactivity of the antibodies (IgG) to rKLi8.3 showed no cross-reactivity with malaria and tuberculosis. In contrast, the KLO8 and rK39 ELISA showed lower specificity and sensitivity in diagnosing VL, and at the same time, increased cross-reactivity with malaria and tuberculosis. The cross-reactivity of rK39 with tuberculosis- and malaria-specific antibodies has also been published.

The available data clearly indicate that for the diagnosis of visceral leishmaniasis in patients from Sudan, the recombinant rKLi8.3 test antigen according to the invention has a much higher sensitivity and specificity than the known test antigens.

In order to also test the performance of the test antigen according to the invention for patients outside of Sudan, sera from India were additionally tested in an ELISA with rKLi8.3, KLO8, and rK39 antigens. Table 3 lists all tested sera with their origins and properties. The results are shown in Table 4. The rKLi8.3 test antigen reacted with all VL patients from India (19 out of 19, i.e., 100%).

TABLE 3
Description of sera from patients with visceral leishmaniasis and controls from India.

| Origin | Number | | | | |
|---|---|---|---|---|---|
| (country) | of sera | Clinical case | | Properties | Classification |
| India | 30 | VL | (n = 19) | The diagnosis is based on the detection of parasites in | Confirmed VL |
| | | | | stained lymph node smears | patients |
| | | EC | (n = 9) | From the same VL endemic area (India) | Negative controls |
| | | MA | (n = 1) | The diagnosis was performed by detecting malaria parasites | |
| | | | | in blood smears | |
| | | TX | (n = 1) | The diagnosis was performed by detecting toxoplasmosis- | |
| | | | | specific antibodies | |

Abbreviations: VL, visceral leishmaniasis; EC, endemic controls; MA, malaria; TX, toxoplasmosis.

TABLE 4
Diagnostic performance of rK39, KLO8, and rKLi8.3 test antigen (ELISA) for visceral leishmaniasis in
India: AUC = area under the curve; TP = correct positive; FN = false negative; TN =
correct negative; FP = false positive; PPV = positive prediction; NPV = negative prediction;
DEV = diagnostic efficiency value. The specificity was calculated using 11 control sera (visceral
leishmaniasis negative), which included 9 endemic control sera, 1 malaria serum, and 1 toxoplasmosis
serum. The sensitivity was calculated using 19 visceral leishmaniasis sera. The values for sensitivity,
specificity, PPV, NPV, and DEV were calculated with a 95% confidence interval. The sera were tested
at 1:800 dilutions and protein concentrations of 5 ng. The cut-off value was set as the mean absorbance
value of the endemic control sera + 3 standard deviations.

| Test antigen | Cut-off | | | | | | | | | | |
|---|---|---|---|---|---|---|---|---|---|---|---|
| (ELISA) | value | AUC | TP | FN | TN | FP | Sensitivity, % | Specificity, % | PPV, % | NPV, % | DEV, % |
| rK39 | 0.146 | 0.9809 | 18 | 1 | 10 | 1 | 94.73 | 90.91 | 94.73 | 90.91 | 93.33 |
| KLO8 | 0.124 | 0.9856 | 18 | 1 | 10 | 1 | 94.73 | 90.91 | 94.73 | 90.91 | 93.33 |
| rKLi8.3 | 0.106 | 0.9904 | 19 | 0 | 10 | 1 | 100 | 90.91 | 95 | 100 | 96.66 |

In order to also test the performance of the test antigen according to the invention on dogs, 332 sera from dogs with canine visceral leishmaniasis (CVL) from Croatia were tested in an ELISA with rKLi8.3, KLO8, and rK39 test antigens. Infection of the dogs (with L. infantum) was confirmed by microscopic analysis of bone marrow aspirates or IFAT.

To determine the suitability and efficiency of the test antigen rKLi8.3 for detecting Leishmania antibodies in canine leishmaniasis, 183 sera from Croatia were analyzed. In addition, 149 sera from uninfected, healthy dogs were tested to determine the cut-off value, which is 0.11 for rKLi8.3 (see Table 5). The ELISA was optimized and performed using the same concentration of test antigen (5 ng/well) and the same serum dilutions (1:800). The ELISA for the analysis of dog sera was performed as described above except for the secondary antibody, since rabbit anti-dog lgG (H+L) (Jackson Immunoresearch Laboratories, Inc.) was used here. The results are shown in Table 6. As has already been shown for humans, the rKLi8.3 test antigen is also very well suited for the serodiagnosis of Leishmania-infected dogs.

TABLE 5
Description of the sera from dogs with canine visceral leishmaniasis and controls from Croatia.

| Origin | Number | | | | |
|---|---|---|---|---|---|
| (country) | of sera | Clinical case | | Properties | Classification |
| Croatia | 332 | CVL | (n = 183) | Indirect immunofluorescence antibody test (IFAT) | Confirmed VL |
| | | | | | dogs |
| | | EC | (n = 149) | From the same VL endemic area (Croatia) | Negative controls |

Abbreviations:
CVL, canine visceral leishmaniasis;
EC, endemic controls;
IFAT, indirect immunofluorescence antibody test

TABLE 6
Diagnostic performance (ELISA) of rK39, KLO8, and rKLi8.3 test antigens in dogs with visceral leishmaniasis
(CVL). The specificity was calculated using 149 visceral leishmaniasis-negative sera from the endemic
area. The sensitivity was calculated using 83 visceral leishmaniasis-positive sera. The values for
sensitivity, specificity, PPV, NPV, and DEV were calculated with a 95% confidence interval. The sera
were tested in a dilution of 1:800 and a protein concentration of 5 ng. The cut-off value was defined
as the mean absorbance value of the endemic control sera + 3 standard deviations.

| Test antigen | Cut-off | | | | | | | | | | |
|---|---|---|---|---|---|---|---|---|---|---|---|
| (ELISA) | value | AUC | TP | FN | TN | FP | Sensitivity, % | Specificity, % | PPV, % | NPV, % | DEV, % |
| KLO8 | 0.14 | 0.9903 | 180 | 3 | 135 | 14 | 98.3 | 90.6 | 92.78 | 97.82 | 94.87 |
| rK39 | 0.15 | 0.9901 | 180 | 3 | 136 | 13 | 98.3 | 91.3 | 93.26 | 97.84 | 95.18 |
| rKLi8.3 | 0.11 | 0.9922 | 181 | 2 | 143 | 6 | 98.9 | 96 | 96.79 | 98.62 | 97.59 |

Abbreviations: CVL, canine visceral leishmaniasis; EC, endemic controls; AUC = area under the curve; TP = correct positive; FN = false negative; TN = correct negative; FP = false positive; PPV = positive prediction; NPV = negative prediction; DEV = diagnostic efficiency value.

Compared to the rK39 test antigen most currently used, the rKLi8.3 test antigen contributes to an improved serodiagnosis of visceral Leishmania infection in humans and dogs, and may thus also provide more precise information about the epidemiological extent and control measures. In addition to the improved diagnostic performance (sensitivity and specificity), serodiagnostics based on rKLi8.3 also offer the advantage of being independent of the pathogen strain (usable in all VL endemic areas) and the infected species, making it suitable for humans and dogs.

The use of the test antigen rKLi8.3 according to the invention results in an improved diagnosis of visceral leishmaniasis, and is therefore an efficient and inexpensive method for monitoring and controlling the spread of this infection.

The test antigen according to the invention was lyophilized and stored in a deep-frozen state. No significant loss of quality could be determined after a storage period of two and a half years. Storage stability is possible if the lyophilized test antigen according to the invention is stored for at least five years.

### FIGURES

FIG. 1 shows the expression, purification, and western blot analysis of the recombinant test antigen (protein) rKLi8.3 according to the invention. The recombinant test antigen rKLi8.3 according to the invention recognizes the positive patient serum, while the negative serum does not react with the recombinant test antigen (protein) rKLi8.3 according to the invention (track 1).

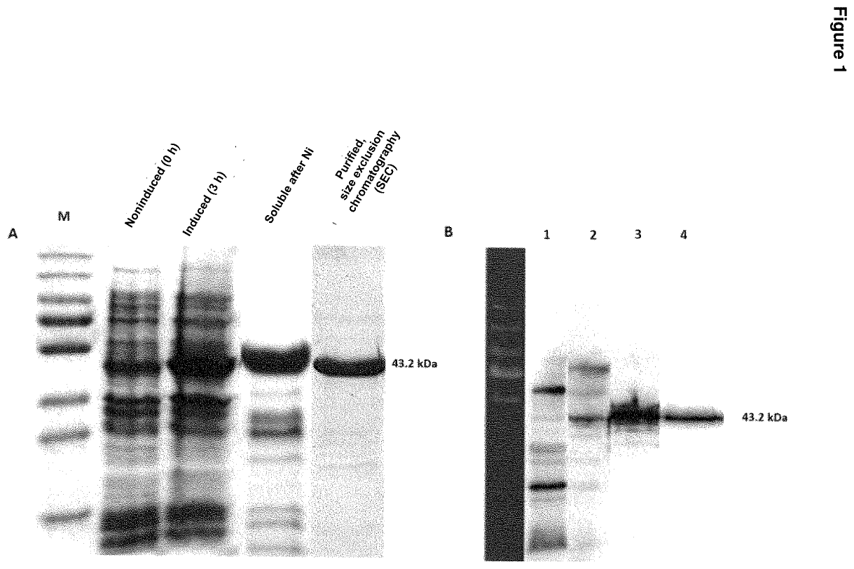

FIG. 2 shows the results of the indirect IgG ELISA for the specific detection of VL. To determine the optimal ELISA conditions, 10 pooled VL sera or 10 pooled healthy control sera in serial two-fold dilution (1:25-1:25600) were titrated against various concentrations of the recombinant test antigen rKLi8.3. A: 50 ng/100 μL, B: 25 ng/100 μL, C: 10 ng/100 μL, D: 5 ng/100 μL. Sera were tested in duplicate, and the mean values were calculated.

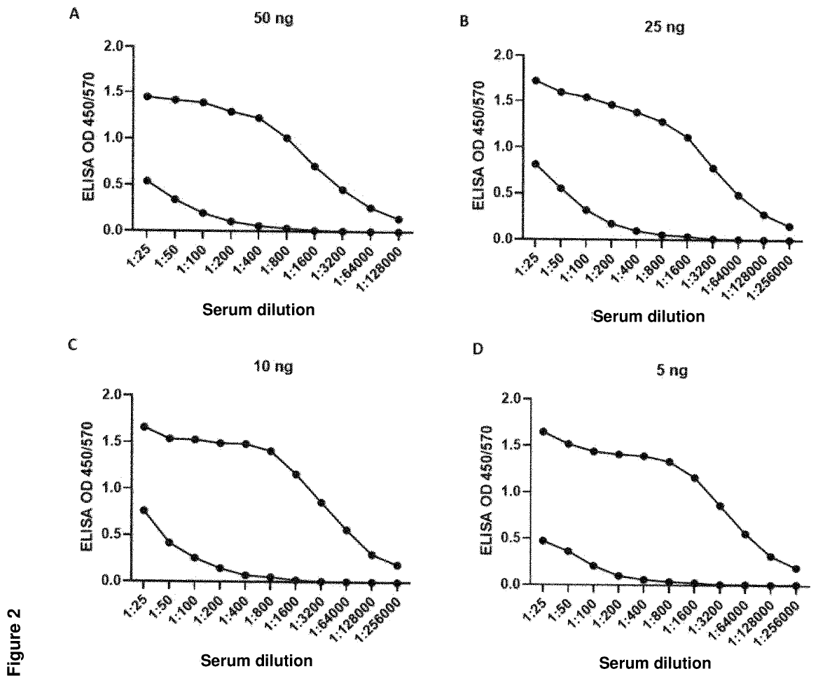

FIG. 3 shows the antibody reaction of human sera (Sudan, Africa) with A: rK39, B: KLO8, and C: rKLi8.3. ELISA plates were coated with recombinant proteins (5 ng/100 μL in 0.1 M sodium carbonate); antibody binding from patients with visceral leishmaniasis (VL) (·, n=172) and various control sera was compared. EC: controls from endemic areas [n=126]; NEC: controls from non-endemic areas [n=10]; MA: malaria patients [n=5]; TB: tuberculosis patients [n=26]. The individual OD values are depicted as dots. The sera were tested at a 1:800 dilution. The symbol “.” represents samples beyond the cut-off range, FN in the VL group and FP in the HC group. The center lines represent the mean values of each group. D: Comparison of the ROC curves of the recombinant proteins. The ROC curves for rKLi8.3, rK39, and KLO8 ELISA were generated using Prism 9.0 software and used to determine the sensitivity, specificity, and AUC for each assay. The y axis represents the sensitivity and the x axis represents the specificity for each assay.

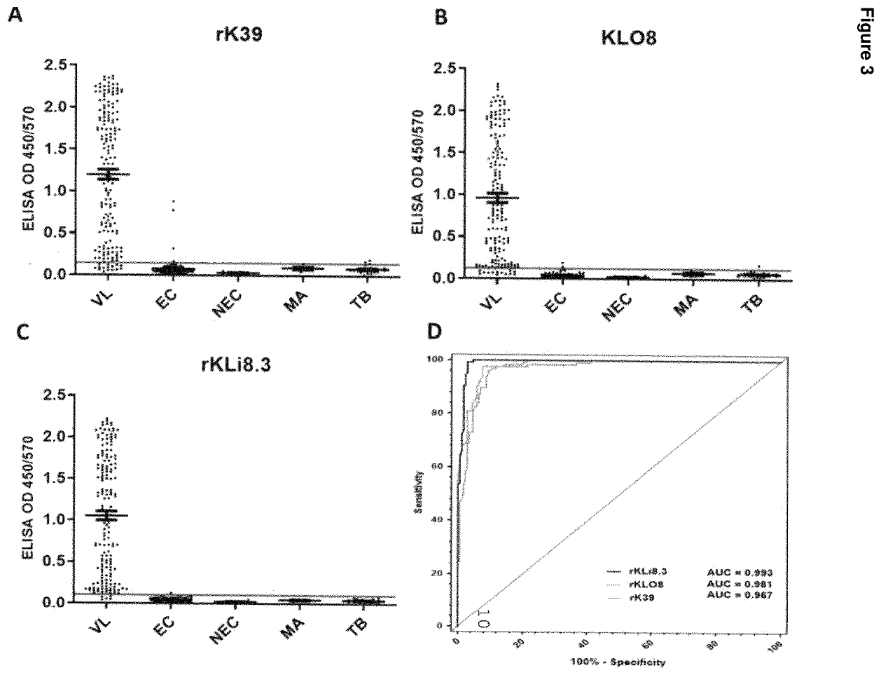

FIG. 4 shows A: rK39, B: KLO8, and C: rKLi8.3 ELISA results with patient sera and control sera from India. The reactivity was checked using sera from VL patients (n=19) or healthy subjects (n=9), malaria (n=1), and toxoplasmosis (n=1). D: Comparison of the ROC curves of the recombinant test antigens. The ROC curves for rKLi8.3, rK39, and KLO8 ELISA were generated using Prism 9.0 software and used to determine the sensitivity, specificity, and AUC for each assay. The y axis represents the sensitivity and the x axis represents the specificity for each assay.

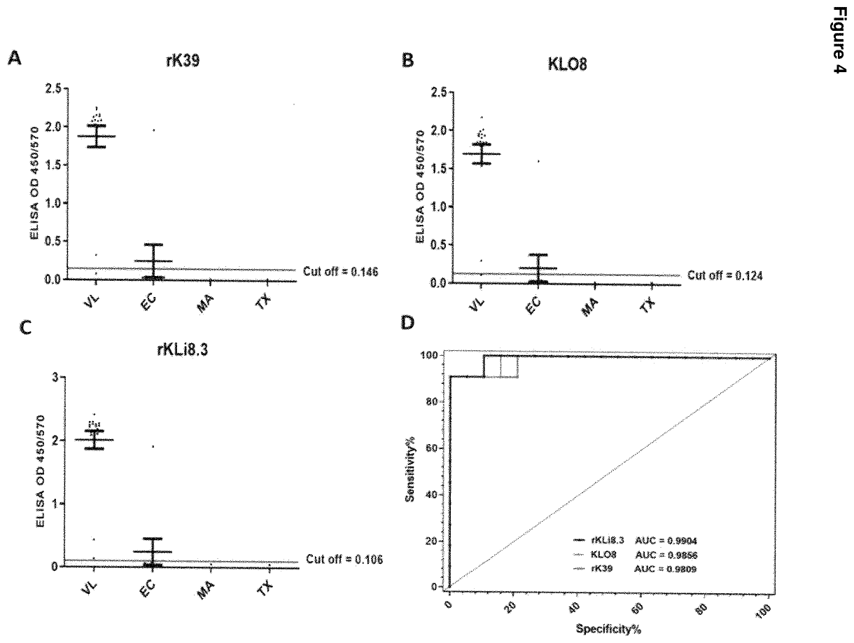

FIG. 5 shows a comparison of the reactivity of dog sera from Croatia and ROC curves. A: rK39, B: KLO8, and C: rKLi8.3 ELISA results with dog and control sera from Croatia. Reactivity was checked using sera from CVL dogs (n=183) or healthy subjects (n=149). D: In the ROC curves, the sensitivity of each test is represented by the y axis and the specificity is represented by the x axis. The ROC curves were generated with Prism 9.0 software and used to determine cut-off, sensitivity, specificity, and area under the curve (AUC) for each assay.

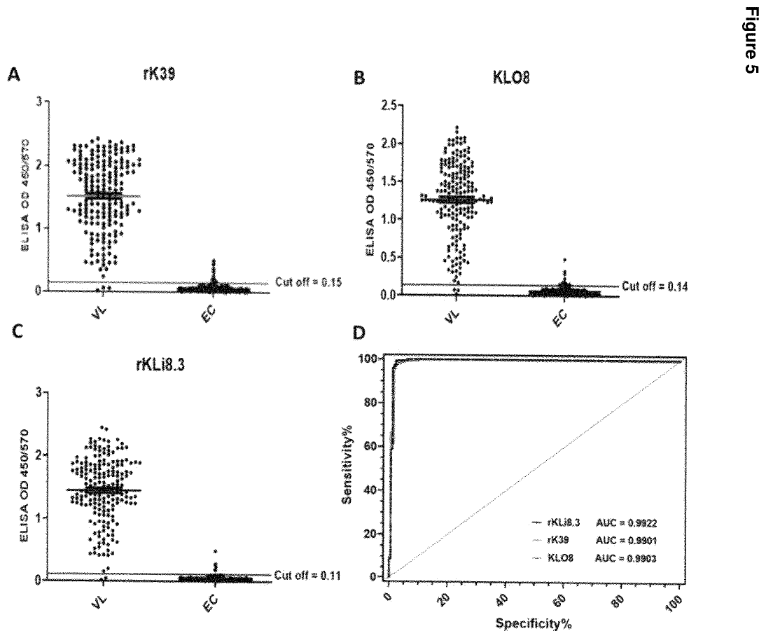

FIG. 6 shows a summary of the kinesin polymorphisms of L. donovani, L. infantum, and L. archibaldi from East Africa and L. donovani from India (KE16), and the differences between the two geographic regions compared to that of L. infantum (L. chagasi) rK39 from Brazil. rK39 is from L. infantum (L. chagasi), KE16 is from L. donovani, KLO8 is from L. donovani, and P=pre-repeat region.

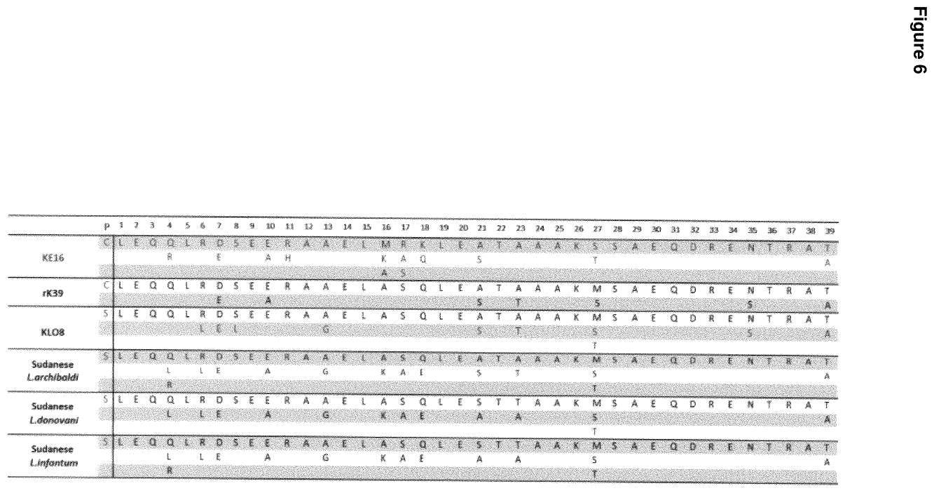

Sequences
SEQ ID NO. 1: Sequence of the forward primer
5′-GAGCTCGCAACCGAGTGGGAGG-3′
SEQ ID NO. 2: Sequence of the reverse primer
5′-GCTCCGCAGCGCGCTCC-3'
SEQ ID NO. 3: DNA sequence KLi8.3
GAGCTCGCAACCGAGTGGGAGGACGCACTCCGCGAGCGTGCGCTTGCAGA
GCGTGACGAAGCCGCTGCAGCCGAACTTGATGCCGCAGCCTCTACTTCCC
AAAACGCACGTGAAAGCGCCTCCGAGCGGCTAACCAGCCTTGAGCAGCTG
CTTCGCGAATCCGAGGGGCGCGCTGCGGAGCTGGCGAGTCAGCTGGAGTC
CACTACTGCTGCGAAGATGTCGGCGGAGCAGGACCGCGAGAACACGAGGG
CCACGCTAGAGCAGCAGCTTCGTGACTCCGAGGAGCGCGCTGCGGAGCTG
GCGAGCCAGCTGGAGGCCACTGCTGCTGCGAAGTCGTCGGCGGAGCAGGA
CCGCGAGAACACGAGGGCCGCGTTGGAGCAGCTGCTTCGCGAATCCGAGG
AGCGCGCTGCGGAGCTGGCGAGCCAGCTGGAGGCCACTGCTGCTGCGAAG
ATGTCGGCGGAGCAGGACCGCGAGAACACGAGGGCCGCGTTGGAGCAGCA
GCTTCGTGACTCCGAGGAGCGCGCTGCGGAGCTGGCGAGTCAGCTGGAGT
CCACTACTGCTGCGAAGATGTGGGGGGAGCAGGACCGCGAGAACACGAGG
GCCACGCTAGAGCAGCAGCTTCGTGACTCCGAGGAGCGCGCTGCGGAGCT
GGCGAGTCAGCTGGAGTCCACTACTGCTGCGAAGACGTCGGGGGAGCAGG
ACCGCGAGAACACGAGGGCCGCGTTGGAGCAGCAGCTTCGTGACTCCGAG
GAGCGCGCTGCGGAGCTGGCGAGCCAGCTGGAGGCCACTGCTGCTGCGAA
GTCGTCGGCGGAGCAGGACCGCGAGAACACGAGGGCCGCGTTGGAGCAGC
AGCTTCGTGACTCCGAGGAGCGCGCTGCGGAGCTGGCGAGCCAGCTGGAG
GCCACTGCTGCTGCGAAGTCGTCGGCGGAGCAGGACCGCGAGAACACGAG
GGCCACGCTAGAGCAGCAGCTTCGTGACTCCGAGGAGCGCGCTGCGGAGC
TGGCGAGTCAGCTGGAGTCCACTACTGCTGCGAAGATGTCGGCGGAGCAG
GACCGCGAGAACACGAGGGCCACGCTAGAGCAGCAGCTTCGTGACTCCGA
GGAGCGCGCTGCGGAGC
SEQ ID NO. 4: Amino acid repeat sequence KLi8.3
LEQLLRESEERAAELASQLESTTAAKMSAEQDRENTRAT
LEQQLRDSEERAAELASQLEATAAAKSSAEQDRENTRAA
LEQLLRESEERAAELASQLEATAAAKMSAEQDRENTRAA
LEQQLRDSEERAAELASQLESTTAAKMSAEQDRENTRAT
LEQQLRDSEERAAELASQLESTTAAKTSAEQDRENTRAA
LEQQLRDSEERAAELASQLEATAAAKSSAEQDRENTRAA
LEQQLRDSEERAAELASQLEATAAAKSSAEQDRENTRAT
LEQQLRDSEERAAELASQLESTTAAKMSAEQDRENTRAT
LEQQLRDSEERAAE
SEQ ID NO. 5: Sequence of the forward primer
for subcloning
5′-GTACATATGGAGCTCGCAACCGAGTGGGAGGACGCA-3′
SEQ ID NO. 6: Sequence of the reverse primer
for subcloning
5′-TACCTCGAGCAGTGTGCTGGAATTCGCCCTTACTCCGCAGG-3′
SEQ ID NO. 7: Nucleotide sequence of recombinant
6xhis-KLi8.3
ATGGGCAGCAGCCATCATCATCATCATCACAGCAGCGGCCTGGTGCCGCG
CGGCAGCCATATGGAGCTCGCAACCGAGTGGGAGGACGCACTCCGCGAGC
GTGCGCTTGCAGAGCGTGACGAAGCCGCTGCAGCCGAACTTGATGCCGCA
GCCTCTACTTCCCAAAACGCACGTGAAAGCGCCTCCGAGCGGCTAACCAG
CCTTGAGCAGCTGCTTCGCGAATCCGAGGAGCGCGCTGCGGAGCTGGCGA
GTCAGCTGGAGTCCACTACTGCTGCGAAGATGTCGGCGGAGCAGGACCGC
GAGAACACGAGGGCCACGCTAGAGCAGCAGCTTCGTGACTCCGAGGAGCG
CGCTGCGGAGCTGGCGAGCCAGCTGGAGGCCACTGCTGCTGCGAAGTCGT
GGGGGGAGCAGGACCGCGAGAACACGAGGGGGGGGTTGGAGCAGCTGCTT
CGCGAATCCGAGGAGCGCGCTGCGGAGCTGGCGAGCCAGCTGGAGGCCAC
TGCTGCTGCGAAGATGTGGGGGGAGCAGGACCGCGAGAACACGAGGGCCG
CGTTGGAGCAGCAGCTTCGTGACTCCGAGGAGCGCGCTGCGGAGCTGGCG
AGTCAGCTGGAGTCCACTACTGCTGCGAAGATGTCGGCGGAGCAGGACCG
CGAGAACACGAGGGCCACGCTAGAGCAGCAGCTTCGTGACTCCGAGGAGC
GCGCTGCGG

1. A method for detecting antibodies against Leishmania in a biological sample, comprising the steps:
(i) providing a biological sample of a human or a dog;
(ii) providing the test antigen, bound to a substrate, containing SEQ ID NO. 8, which has 8.3 repetitive sequences and which can be recognized and bound by antibodies against various Leishmania strains;
(iii)) incubating the biological sample from step (i) with the test antigen from step (ii), so that antigen-antibody complexes are formed if the biological sample from step (i) contains antibodies against at least one Leishmania strain;
(iv) removing antibodies and other material, not bound to the test antigen from step (ii), from the biological sample from step (i) in a first washing step;
(v) adding a human-specific secondary antibody if the biological sample from step (i) comes from a human, or adding a canine-specific secondary antibody if the biological sample from step (i) comes from a dog, wherein the respective secondary antibody is detectable using an agent for visualizing binding of the secondary antibody, so that the respective secondary antibody binds to the antigen-antibody complexes from step (iii);
(vi) removing unbound secondary antibodies from step (v) in a second washing step;
(vii) visualizing the binding of the secondary antibody to the antigen-antibody complexes from step (iii) by adding a suitable agent.

2. The method according to claim 1, characterized in that the biological sample is serum, plasma, whole blood, lymph node aspirate, saliva, tissue fluid, cerebrospinal fluid, urine, or some other body fluid.

3. The method according to claim 1, characterized in that the means for detecting the binding between the secondary antibody and the antigen-antibody complexes from step (v) is an enzyme that causes a color change with a suitable substrate.

4. The method according to claim 1, characterized in that the means for detecting the binding between the secondary antibody and the antigen-antibody complexes from step (v) induces a color reaction.

5. The method according to claim 1, characterized in that the means for detecting binding between the secondary antibody and the antigen-antibody complexes from step (v) is gold colloid.

6. The method according to claim 1, characterized in that antibodies against Leishmania are detected, selected from the group Leishmania chagasi, Leishmania donovani, and Leishmania infantum.

7. The method according to claim 1, characterized in that the specificity is at least 90% and the sensitivity is at least 97%.

8. A test kit for carrying out the method according to claim 1, characterized in that the test kit includes at least:
a substrate suitable for binding the test antigen;
a test antigen, bound to this substrate, with SEQ ID NO. 8, which has 8.3 repetitive sequences and which can be recognized and bound by antibodies against various Leishmania strains and forms antigen-antibody complexes;
a human-specific secondary antibody if the biological sample is from a human, or a canine-specific secondary antibody if the biological sample is from a dog;
a means for detecting the binding between the secondary antibody and the antigen-antibody complexes.

9. The test kit according to claim 8, characterized in that the substrate is a microtiter plate, a test field, or a test strip.

10. Production of the test antigen according to SEQ ID NO. 8, which has 8.3 repetitive sequences and which can be recognized and bound by antibodies against various Leishmania strains, comprising the steps:
(i) providing promastigote Leishmania;
(ii) isolating the genomic DNA from the promastigote Leishmania from step (i);
(iii) amplifying the gene segment from the DNA from step (ii) according to SEQ ID NO. 3 with the forward PCR primer according to SEQ ID NO. 1 and the reverse PCR primer according to SEQ ID NO. 2, resulting in an amplification product;
(iv) isolating and purifying the amplification product from step (iii);
(v) cloning the purified amplification product from step (iv) into a vector;
(vi) transfecting competent E. coli with the vector from step (v);
(vii) expressing the test antigen in E. coli;
(viii) lysing the E. coli from step (vii);
(ix) isolating the test antigen from the lysed E. coli from step (viii);
(x) purifying the test antigen from step (ix).

11. A test antigen with SEQ ID NO. 8 that is produced by the method according to claim 10.

12. The test antigen according to claim 10, characterized in that the test antigen has a storage stability of at least two and a half years in lyophilized form.

13. The test antigen according to claim 10, characterized in that the test antigen has a storage stability of at least five years in lyophilized form.

14. Use of the test antigen according to claim 10.

15. Use of the test antigen according to claim 10 in a test kit.


SEQUENCE LISTING (15 sequences)

SEQ ID NO: 1 - DNA, 22 bp, synthetic construct
  misc_feature 1..22: note = Forward Primer putative kinesin K39 Leishmania infantum
  source 1..22: mol_type = other DNA
gagctcgcaaccgagtgggagg

SEQ ID NO: 2 - DNA, 17 bp, synthetic construct
  misc_feature 1..17: note = Reverse primer putative kinesin K39 Leishmania infantum
  source 1..17: mol_type = other DNA
gctccgcagcgcgctcc

SEQ ID NO: 3 - DNA, 1117 bp, synthetic construct
  misc_feature 1..1117: note = DNA Sequence KLi8.3
  source 1..1117: mol_type = other DNA
gagctcgcaaccgagtgggaggacgcactccgcgagcgtgcgcttgcagagcgtgacgaagccgctgcagccgaacttgatgccgcagcctctacttcccaaaacgcacgtgaaagcgcctccgagcggctaaccagccttgagcagctgcttcgcgaatccgaggagcgcgctgcggagctggcgagtcagctggagtccactactgctgcgaagatgtcggcggagcaggaccgcgagaacacgagggccacgctagagcagcagcttcgtgactccgaggagcgcgctgcggagctggcgagccagctggaggccactgctgctgcgaagtcgtcggcggagcaggaccgcgagaacacgagggccgcgttggagcagctgcttcgcgaatccgaggagcgcgctgcggagctggcgagccagctggaggccactgctgctgcgaagatgtcggcggagcaggaccgcgagaacacgagggccgcgttggagcagcagcttcgtgactccgaggagcgcgctgcggagctggcgagtcagctggagtccactactgctgcgaagatgtcggcggagcaggaccgcgagaacacgagggccacgctagagcagcagcttcgtgactccgaggagcgcgctgcggagctggcgagtcagctggagtccactactgctgcgaagacgtcggcggagcaggaccgcgagaacacgagggccgcgttggagcagcagcttcgtgactccgaggagcgcgctgcggagctggcgagccagctggaggccactgctgctgcgaagtcgtcggcggagcaggaccgcgagaacacgagggccgcgttggagcagcagcttcgtgactccgaggagcgcgctgcggagctggcgagccagctggaggccactgctgctgcgaagtcgtcggcggagcaggaccgcgagaacacgagggccacgctagagcagcagcttcgtgactccgaggagcgcgctgcggagctggcgagtcagctggagtccactactgctgcgaagatgtcggcggagcaggaccgcgagaacacgagggccacgctagagcagcagcttcgtgactccgaggagcgcgctgcggagc

SEQ ID NO: 4 - protein, 326 aa, synthetic construct
  REGION 1..326: note = Amino Acid-Repeat-Sequence KLi8.3
LEQLLRESEERAAELASQLESTTAAKMSAEQDRENTRATLEQQLRDSEERAAELASQLEATAAAKSSAEQDRENTRAALEQLLRESEERAAELASQLEATAAAKMSAEQDRENTRAALEQQLRDSEERAAELASQLESTTAAKMSAEQDRENTRATLEQQLRDSEERAAELASQLESTTAAKTSAEQDRENTRAALEQQLRDSEERAAELASQLEATAAAKSSAEQDRENTRAALEQQLRDSEERAAELASQLEATAAAKSSAEQDRENTRATLEQQLRDSEERAAELASQLESTTAAKMSAEQDRENTRATLEQQLRDSEERAAE

SEQ ID NO: 5 - DNA, 36 bp, synthetic construct
  misc_feature 1..36: note = Forward Primer for DNA-Construct pCR2.1/KLi8.3
  source 1..36: mol_type = other DNA
gtacatatggagctcgcaaccgagtgggaggacgca

SEQ ID NO: 6 - DNA, 41 bp, synthetic construct
  misc_feature 1..41: note = Reverse primer for DNA-Construct pCR2.1/KLi8.3
  source 1..41: mol_type = other DNA
tacctcgagcagtgtgctggaattcgcccttactccgcagc

SEQ ID NO: 7 - DNA, 1182 bp, synthetic construct
  misc_feature 1..1182: note = Nucleotide sequence of recombinant 6xhis-KLi8.3 for his-KLi8.3 fusion protein
  source 1..1182: mol_type = other DNA
atgggcagcagccatcatcatcatcatcacagcagcggcctggtgccgcgcggcagccatatggagctcgcaaccgagtgggaggacgcactccgcgagcgtgcgcttgcagagcgtgacgaagccgctgcagccgaacttgatgccgcagcctctacttcccaaaacgcacgtgaaagcgcctccgagcggctaaccagccttgagcagctgcttcgcgaatccgaggagcgcgctgcggagctggcgagtcagctggagtccactactgctgcgaagatgtcggcggagcaggaccgcgagaacacgagggccacgctagagcagcagcttcgtgactccgaggagcgcgctgcggagctggcgagccagctggaggccactgctgctgcgaagtcgtcggcggagcaggaccgcgagaacacgagggccgcgttggagcagctgcttcgcgaatccgaggagcgcgctgcggagctggcgagccagctggaggccactgctgctgcgaagatgtcggcggagcaggaccgcgagaacacgagggccgcgttggagcagcagcttcgtgactccgaggagcgcgctgcggagctggcgagtcagctggagtccactactgctgcgaagatgtcggcggagcaggaccgcgagaacacgagggccacgctagagcagcagcttcgtgactccgaggagcgcgctgcggagctggcgagtcagctggagtccactactgctgcgaagacgtcggcggagcaggaccgcgagaacacgagggccgcgttggagcagcagcttcgtgactccgaggagcgcgctgcggagctggcgagccagctggaggccactgctgctgcgaagtcgtcggcggagcaggaccgcgagaacacgagggccgcgttggagcagcagcttcgtgactccgaggagcgcgctgcggagctggcgagccagctggaggccactgctgctgcgaagtcgtcggcggagcaggaccgcgagaacacgagggccacgctagagcagcagcttcgtgactccgaggagcgcgctgcggagctggcgagtcagctggagtccactactgctgcgaagatgtcggcggagcaggaccgcgagaacacgagggccacgctagagcagcagcttcgtgactccgaggagcgcgctgcggagtaa

SEQ ID NO: 8 - protein, 393 aa, synthetic construct
  REGION 1..393: note = Amino acid sequence of recombinant 6xhis-rKLi8.3 for purified rKLi8.3 protein
MGSSHHHHHHSSGLVPRGSHMELATEWEDALRERALAERDEAAAAELDAAASTSQNARESASERLTSLEQLLRESEERAAELASQLESTTAAKMSAEQDRENTRATLEQQLRDSEERAAELASQLEATAAAKSSAEQDRENTRAALEQLLRESEERAAELASQLEATAAAKMSAEQDRENTRAALEQQLRDSEERAAELASQLESTTAAKMSAEQDRENTRATLEQQLRDSEERAAELASQLESTTAAKTSAEQDRENTRAALEQQLRDSEERAAELASQLEATAAAKSSAEQDRENTRAALEQQLRDSEERAAELASQLEATAAAKSSAEQDRENTRATLEQQLRDSEERAAELASQLESTTAAKMSAEQDRENTRATLEQQLRDSEERAAE

SEQ ID NO: 9 - DNA, 18 bp, synthetic construct
  misc_feature 1..18: note = His Tag
  source 1..18: mol_type = other DNA
catcatcatcatcatcac

SEQ ID NO: 10 - protein, 40 aa, Leishmania donovani
  REGION 1..40: note = Pre-repeat region
CLEQQLRDSEERAAELMRKLEATAAAKSSAEQDRENTRAT

SEQ ID NO: 11 - protein, 40 aa, Leishmania infantum
  REGION 1..40: note = Pre-repeat region
CLEQQLRDSEERAAELASQLEATAAAKMSAEQDRENTRAT

SEQ ID NO: 12 - protein, 40 aa, Leishmania donovani
  REGION 1..40: note = Pre-repeat region
SLEQQLRDSEERAAELASQLEATAAAKMSAEQDRENTRAT

SEQ ID NO: 13 - protein, 40 aa, Leishmania donovani; identical to SEQ ID NO: 12
  REGION 1..40: note = Pre-repeat region
  source 1..40: note = Leishmania donovani archibaldi

SEQ ID NO: 14 - protein, 40 aa, Leishmania donovani
  REGION 1..40: note = Pre-repeat region
SLEQQLRDSEERAAELASQLESTTAAKMSAEQDRENTRAT

SEQ ID NO: 15 - protein, 40 aa, Leishmania infantum; identical to SEQ ID NO: 14
  REGION 1..40: note = Pre-repeat region